(19) United States
(12) Patent Application Publication
(10) Pub. No.: US 2005/0287114 A1
(43) Pub. Date: Dec. 29, 2005

(54) Water-soluble polymeric bone-targeting drug delivery system

(75) Inventors: Dong Wang, Omaha, NE (US); Scott C. Miller, Salt Lake City, UT (US); Pavla Kopeckova, Salt Lake City, UT (US); Jindrich Kopecek, Salt Lake City, UT (US)

Correspondence Address: THORPE NORTH & WESTERN, LLP., 8180 SOUTH 700 EAST, SUITE 200, SANDY, UT, 84070, US

(73) Assignee: University of Utah Research Foundation

(21) Appl. No.: 11/150,865

(22) Filed: Jun. 10, 2005

Related U.S. Application Data
(63) Continuation of application No. PCT/US04/00276, filed on Jan. 6, 2004.
(60) Provisional application No. 60/438,430, filed on Jan. 6, 2003.

(51) Int. Cl. A61K 38/08; A61K 31/785; C07C 237/26

(52) U.S. Cl. 424/78.27; 525/54.1; 525/296; 552/205; 514/152

(57) ABSTRACT

The present invention provides bone-targeting polymeric drug delivery systems based on HPMA and related copolymers and methods of making thereof. The water-soluble bone-targeting polymeric conjugates of the present invention comprise water-soluble copolymer backbones (P) which are linked, via a first spacer (S₁), with a bone-related therapeutic agents or drug (D) and, via a second spacer (S₂), with a bone-targeting moiety (T).

### BACKGROUND OF THE INVENTION

1. Field of the Invention

The present invention relates generally to bone-targeting drug delivery systems. More particularly, the present invention relates to water-soluble polymeric bone-targeting drug delivery systems based on copolymers of N-(2-hydroxypropyl) methacrylamide and other functionally related monomers.

2. Related Art

Bone is a highly specialized form of connective tissue which provides an internal support system in all higher vertebrates. It is a complex living tissue in which the extracellular organic matrix is mineralized, conferring marked rigidity and strength to the skeleton while still maintaining some degree of elasticity. In addition to its supportive and protective functions, bone is the major source of inorganic ions, actively participating in calcium homeostasis in the body. Marks, S. C. Jr. & Odgren, P. R. (2002) Principles of Bone Biology, 2nd Edition (Bilezikian, J. P., Raisz, L. G., Rodan, G. A., Ed.) pp 3-15. To maintain its normal function, bone is continuously being resorbed and rebuilt throughout the skeleton. Resorption is carried out by hematopoietically derived osteoclasts, whereas the rebuilding of lost bone is by osteoblasts, which are derived from bone marrow stromal cells. In healthy individuals, bone resorption and formation are well balanced with the bone mass being maintained in a steady state. Any disturbance of this balance may lead to a number of bone diseases, such as osteoporosis, Paget's disease, osteopetrosis, bone cancer, etc. Odgren, P. R. & Martin, T. J. (2000) Science 289,1508-1514. Over the past decade, people's understanding of bone biology has improved greatly. The transcription factor, core binding factor 1 (Cbfa1), has been identified as being specifically expressed in cells of osteoblast linage, and plays a major role in osteoblast differentiation. Ducy, P. et al.(1997) Cell 89, 747-754.

It has been shown that osteoblasts/stromal cells express the two molecules that are essential and sufficient to promote osteoclastogenesis: macrophage colony-stimulating factor (M-CSF) and the receptor for activation of nuclear factor kappa B (NF-κB) ligand (RANKL). Besides these factors, which regulate the number of osteoclasts, other molecules have been identified as being important for the normal function of osteoclasts. For example, αvβ3 integrin has been found to be responsible for the formation of the sealing zone and the transduction of bone matrix derived signals, which are pivotal to bone resorption. A vacuolar H⁺-adenosine triphosphatase (H⁺-ATPase) and carbonic anhydrase II (CA2) are believed to be critical in the maintenance of a lower pH in resorption lacuna, which are responsible for the dissolution of the inorganic bone matrix. The remaining demineralized bone matrix (type I collagen, >90%) is mainly digested by a newly found lysosomal cysteine protease, cathepsin K, which shows its highest expression in osteoclasts. Takahashi, N. et al.(2002) Principles of Bone Biology, 2nd Edition (Bilezikian, J. P., Raisz, L. G., Rodan, G. A., Ed.), pp 109-126; V\ddot{aa}nänen, K. & Zhao, H. (2002) Principles of Bone Biology, 2nd Edition (Bilezikian, J. P., Raisz, L. G., Rodan, G. A., Ed.), pp 127-139.

Many of the molecules mentioned above have been listed as novel therapeutic targets for the treatment of bone diseases. OPG, cathepsin K inhibitors, CA2 inhibitors, αvβ3 antagonists, and c-Src homology 2 inhibitors have been studied for their antiresorptive activity. Prostaglandin E1 & E2, prostaglandin E EP4 receptor agonists, statins [inhibitors of hydroxy-methyl-glutaryl-CoA (HMG-CoA) reductase], parathyroid hormone (PTH), and growth factors (including TGF-b, FGFs and the BMPs) have been considered for stimulation of bone formation. Gene therapy has also been tried for the prevention and treatment of bone disease. Capparelli, C. et al.(2000) Cancer Res. 60, 783-787; Yamashita, D. S. & Dodds, R. A. (2000) Curr. Pharm. Des. 6, 1-24; Minkin, C. & Jennings, J. M. (1972) Science 176, 1031-1033; Engleman, V. W. et al. (1 997) J Clin. Invest. 99, 2284-2292; Shakespeare, W. et al. (2000) Proc. Natl. Acad. Sci. 97, 9373-9378; Yoshida, K. et al. (2002) Proc. Natl. Acad. Sci. 99, 4580-4585; Mundy, G. et al. (1999) Science 286, 1946-1949; Lindsay, R. & Nieves, J. (1997) Lancet 350, 550-555.

However, most of these therapeutic agents are not specifically targeted to bone, which greatly hampers their clinical application in the treatment of bone diseases. The recent reports on the long-term effects of hormone replacement therapy (HRT) clearly demonstrate how tragic it can be if therapeutic agents are not specifically delivered to their target. Writing Group for the Women's Health Initiative Investigation. (2002) J Am. Med. Assoc. 288, 321-333; Lacey, J. V. Jr et al J. Am. Med. Assoc. 288, 334-341. A few attempts have been made to target drugs to hard tissue. Tetracycline (TC) and its analogs can be linked to different drugs to increase their bone-seeking ability. Pierce, W. et al. (984) Proc. Soc. Exp. Bio. Med. 186, 96-102; Orme, M. W., Labroo, V. M. (1994) Bioorg. Med. Chem. Lett. 4, 1375- 1380; Wilson, T. M et al. (1996) Bioorg. Med. Chem. Lett. 6, 1043-1046. Bisphosphonates have been conjugated to different macromolecules (proteins, PEG) and low molecular weight compounds to make them osteotropic. Bentz, H. & Rosen, D. (1992) EP 0 512 844 A1; Uludag, H. & Yang, J. (2002) Biotechnol. Prog. 18, 604-611; Verbeke, K. et al. (2002) Bioconjugate Chem. 13, 16-22. Recently, glutamic acid and aspartic acid peptides have been reported to be useful as bone-targeting moieties to deliver drugs to bone. Kasugai, S. et al. (2000) J. Bone Miner. Res. 15, 936-943. However, such pharmaceutical research has been limited and has lagged behind people's understanding of bone biology (see FIG. 1 for chemical structures of some molecules with strong bone affinity).

### SUMMARY OF THE INVENTION

It has been recognized that it would be advantageous to develop a water-soluble polymeric conjugate for bone-targeted drug delivery with improved pharmacokinetic parameters and better water solubility of the loaded drugs.

The present invention provides a water-soluble, polymeric conjugate for bone-targeted drug delivery. More specifically, the water-soluble bone-targeting polymeric conjugate of the present invention comprises a water-soluble copolymer backbone (P) which is linked, via a first spacer (S₁), with a bone-related therapeutic agent or drug (D) and, via a second spacer (S₂), with a bone-targeting moiety (T), and wherein said copolymer comprises 5.0 to 99.0 mol% of monomeric units comprising N-(2-hydroxypropyl)methacrylamide (HPMA) and other functionally related monomers. More specifically, such monomers can be one or more members selected from the group including, but not limited to, N-(2-hydroxypropyl)methacrylamide, N-(2-hydroxyethyl)methacrylamide, N-isopropylacrylamide, acrylamide, N,N-dimethylacrylamide, N-vinylpyrrolidone, vinyl acetate (the resulting polymer can be hydrolyzed into polyvinyl alcohol, commonly known as PVA), 2-methacryloxyethyl glucoside, acrylic acid, methacrylic acid, vinylphosphonic acid, styrenesulfonic acid, maleic acid, 2-methacryloxyethyltrimethylammonium chloride, methacrylamidopropyltrimethylammonium chloride, methacryloylcholine methyl sulfate, N-methylolacrylamide, 2-hydroxy-3-methacryloxypropyltrimethylammonium chloride, 2-methacryloxyethyltrimethylammonium bromide, 2-vinyl-1-methylpyridinium bromide, 4-vinyl-1-methylpyridinium bromide, ethyleneimine, (N-acetyl)ethyleneimine, (N-hydroxyethyl)ethyleneimine and allylamine.

The present invention also relates to a pharmaceutical composition comprising the bone targeting therapeutic copolymer of the present invention. The pharmaceutical composition may be formulated for oral administration, inhalation, implantation (of the drug containing depot) and injection (systemic or local).

The present invention further includes a novel compound composed of a tetracycline derivative, 9-Gly-ATC (illustrated in FIG. 2), which can be used as a bone-targeting moiety and as a novel antibiotic agent, and a process for the manufacture thereof.

Additional features and advantages of the invention will be apparent from the detailed description which follows, taken in conjunction with the accompanying drawings, which together illustrate, by way of example, features of the invention.

### BRIEF DESCRIPTION OF THE DRAWINGS

FIG. 1 illustrates the chemical structures of selected bone-targeting moieties;

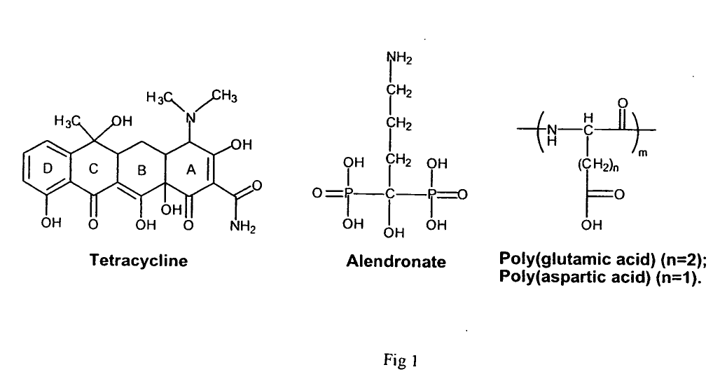

FIG. 2 illustrates the synthetic scheme of 9-amino-anhydrotetracycline (9-Gly-ATC) according to the present invention;

FIG. 3 illustrates the chemical structures of representative bone-targeting copolymeric conjugates of the present invention;

FIG. 4 illustrates the binding effects of polymeric bone-targeting conjugates of the present invention to hydroxyapatite; and

FIG. 5 illustrates, by means of fluorescent markers, the in vivo binding of the polymeric conjugates of the present invention to bone. (A) Saline, no autofluorescence observed in the bone; (B) P-FITC, no FITC label observed in the bone; (C) P-Alendronate-FITC, endosteal surfaces labeled with FITC; (D) P-Alendronate-FITC, endosteum and periosteum of diaphyseal shaft labeled with FITC; (E) P-D-Asp₈-FITC, primary spongiosa and endosteal surfaces labeled with FITC; (F) P-D-Asp₈-FITC, endosteum of diaphyseal shaft labeled with FITC.

### DETAILED DESCRIPTION

Reference will now be made to the exemplary embodiments and specific language will be used herein to describe the same. It will nevertheless be understood that no limitation of the scope of the invention is thereby intended. Alterations and further modifications of the inventive features illustrated herein, and additional applications of the principles of the invention as illustrated herein, which would occur to one skilled in the relevant art and having possession of this disclosure, are to be considered within the scope of the invention.

It must be noted that, as used in this specification and the appended claims, the singular forms “a,” “an,” and “the” include plural referents unless the context clearly dictates otherwise. Thus, for example, reference to a copolymer grafted with “a bone-targeting moiety” includes reference to two or more of such moieties, and reference to “a bone therapeutic agent or drug” includes reference to two or more of such agents or drugs.

In describing and claiming the present invention, the following terminology will be used in accordance with the definitions set out below. As used herein, the term “bone related therapeutic agent or drug” or any other similar term means any chemical or biological material or compound suitable for administration by methods previously known in the art and/or by the methods taught in the present invention and that induce a desired biological or pharmacological effect. Such effects may include but are not limited to (1) having a prophylactic effect on bone and preventing an undesired biological effect such as preventing an infection, (2) alleviating a condition caused by a disease, for example, alleviating pain or inflammation caused as a result of disease, and/or (3) either alleviating, reducing, or completely eliminating a disease from bone.

As used herein, the term “biodegradable” or “biodegradation” is defined as the conversion of materials into less complex intermediates or end products by solubilization hydrolysis under physiological conditions, or by the action of biologically formed entities which can be enzymes or other products of the organism.

As used herein, the term “non-degradable” refers to a chemical structure that cannot be cleaved under physiological condition, even with any external intervention.

As used herein, the term “degradable” refers to the ability of a chemical structure to be cleaved via physical (such as ultrasonication), chemical (such as pH of less than 4 or more than 9) or biological (enzymatic) means.

As used herein, the term “biocompatible” means materials, or the intermediates or end products of materials, formed by solubilization hydrolysis, or by the action of biologically formed entities which can be enzymes or other products of the organism and which cause no adverse effects on the body.

As used herein, the term “cross-links” or “cross-linkage” refers to a chemical bridge with a molecular weight much less than the molecular weight of the two polymer chains being joined together.

As used herein, the term “water-soluble” refers to the capability of being completely dissolved in an aqueous solution under possible physiological conditions in vivo; and capable of being completely dissolved in an aqueous solution under in vitro conditions of 1-50° C., with a pH value between 2 and 10.

As used herein, “aryl” means an aromatic structure which includes but not limited to: benzenoid and its derivatives, heteroyclic aromatic compounds such as pyridine, pyrrole, furan thiophene, purine, pyrimidine and their derivatives.

As used herein, the term “bone-targeting” refers to the capability of preferentially accumulating in hard tissue rather than any other organ or tissue, after administration in vivo.

As used herein, “effective amount” means the amount of a bioactive agent that is sufficient to provide the desired local or systemic effect and performance at a reasonable risk/benefit ratio as would attend any medical treatment.

As used herein, “administering” and similar terms means delivering the composition to the individual being treated such that the composition is capable of being circulated systemically or distributed locally at the desired sites. Preferably, the compositions of the present invention are administered by oral, subcutaneous, intramuscular, transdermal, transmucosal, intravenous, or intraperitoneal routes. In addition, intraarticular, intraperiodontal or any other possible local injections routes are also included. Injectables for such use can be prepared in conventional forms, either as a liquid solution or suspension, or in a solid form that is suitable for preparation as a solution or suspension in a liquid prior to injection, or as an emulsion. Suitable excipients that can be used for administration include, for example, water, saline, dextrose, glycerol, ethanol, and the like; and if desired, minor amounts of auxiliary substances such as wetting or emulsifying agents, buffers, and the like. For oral administration, it can be formulated into various forms such as solutions, tablets, capsules, etc.

One aspect of the present invention provides a water-soluble bone-targeting drug delivery system comprising an inert synthetic polymeric carrier combined through degradable or non-degradable spacers with a bone-related therapeutic agent and with a bone-targeting moiety. Optionally the system also contains an optional bioassay label and an optional cross-linkage. The system may be represented by the following formula:

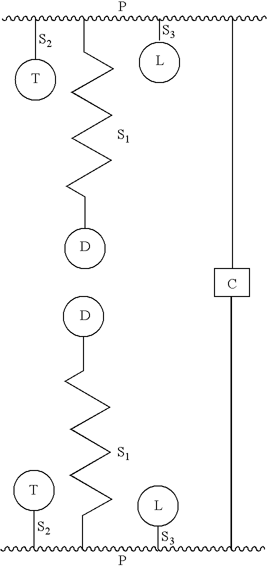

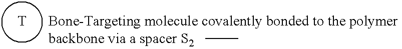

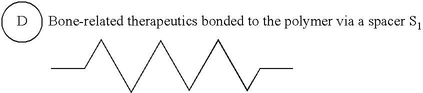

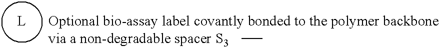

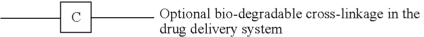

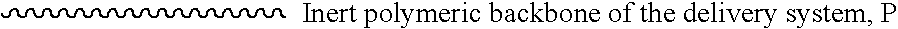

wherein D is a bone-related therapeutic agent bonded to a water soluble inert polymer backbone (P) via a first spacer (S₁) which may be biodegradable or non-biodegradable; T is a bone-targeting molecule covalently bound to the polymer backbone (P) via a biodegradable or non-degradable spacer (S₂); L is an optional bio-assay label covalently bonded to the polymer backbone (P) via a non-degradable third spacer (S₃) which can be the same or different than S₁ or S₂ when they are non-degradable; and C is an optional biodegradable cross-linkage between two polymer chains (P).

More specifically, the water-soluble bone-targeting polymeric conjugate of the present invention comprises a water-soluble copolymer backbone (P) which is linked, via a first spacer (S₁), with a bone-related therapeutic agent or drug (D) and, via a second spacer (S₂), with a bone-targeting moiety (T), and wherein said copolymer comprises 5.0 to 99.0 mol% of monomeric units selected from the group including but not limited to N-(2-hydroxypropyl)methacrylamide, N-(2-hydroxyethyl)methacrylamide, N-isopropylacrylamide, acrylamide, N,N-dimethylacrylamide, N-vinylpyrrolidone, vinyl acetate (the resulting polymer can be hydrolyzed into polyvinyl alcohol, commonly known as PVA), 2-methacryloxyethyl glucoside, acrylic acid, methacrylic acid, vinylphosphonic acid, styrenesulfonic acid, maleic acid, 2-methacryloxyethyltrimethylammnonium chloride, methacrylamidopropyltrimethylammonium chloride, methacryloylcholine methyl sulfate, N-methylolacrylamide, 2-hydroxy-3-methacryloxypropyltrimethylammonium chloride, 2-methacryloxyethyltrimethylammonium bromide, 2-vinyl-1-methylpyridinium bromide, 4-vinyl-1-methylpyridinium bromide, ethyleneimine, (N-acetyl)ethyleneimine, (N-hydroxyethyl)ethyleneimine and allylamine.

The first spacer (S₁) may be biodegradable or non-biodegradable. The second spacer (S₂) may also be biodegradable or non-biodegradable and may be the same as or different than the first spacer (S₁). Optionally, a bioassay label (L) may be attached to the copolymer backbone via a non-degradable third spacer (S₃) which can be the same as or different than S₁ and S₂ when they are non-degradable. The bone targeting moiety and bone-related therapeutic agent containing copolymeric conjugates of the present invention may also be optionally cross-linked via a biodegradable cross-linkage (C).

In accordance with more detailed aspects of the present invention, the molecular weight of the copolymer backbone (P) is within the range of 1 to 500 kDa.

The spacer (S₁) between the bone-related therapeutic agent and the copolymeric backbone may be a biodegradable structure, which includes but is not limited to the following:
A. Peptide structure: 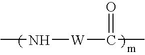
wherein W is the portion of an amino acid other than an NH₂ or COOH group, said amino acid having an L-configuration and being selected from all the essential amino acids, and m is an integer from 1 to 10.
B. Structures that can proceed to 1,6 elimination, e.g. 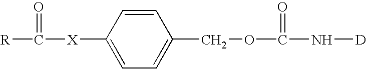
wherein R may be a peptide structure as same as the structure A described above, which is directly connected to the polymer backbone and D represents the bone-related therapeutic agent of which the amine group(-NH-) is a part; and X can be O or NH.
C. A pH sensitive structure that can be cleaved under acidic conditions, such as the cis-aconityl spacer group: 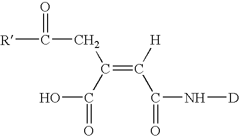
wherein R′ may be a C₀ to C₁₀ alkyl amino, aryl amino, a C₀ to C₁₀ alkyl amino or aryl oxy, which is directly connected to the polymer backbone and D represents the bone-related therapeutic agent of which the amine group(-NH-) is a part.

The spacer (S₁) between the bone-related therapeutic agent and the polymeric backbone may also be non-degradable and can be a covalent bond or any other chemical structure which cannot be cleaved under physiological environments or conditions.

The water-soluble polymeric bone-targeting drug delivery systems, based on copolymers of N-(2-hydroxypropyl)methacrylamide (HPMA) or any of the other listed related monomers, of the present invention may be used as universal vehicles for the specific delivery of bone related therapeutic agents. Theoretically, any bone therapeutic agent can be covalently loaded onto these delivery systems via spacers as described above. Other benefits of these conjugates are greatly improved pharmacokinetic parameters and better water solubility of the loaded drugs.

The bone-related therapeutic agents suitable for the present invention include but are not limited to: inhibitors (such as cathepsin K inhibitor, metalloproteinase inhibitors), agonists (such as prostaglandin E receptor agonist), antagonists (such as aαvβ3 antagonist), and anabolic agents (such as prostaglandin E1 or E2 and its analogs; parathyroid hormone; statins), therapeutic peptides or proteins (such as hormones and cytokines), and therapeutic metal ions. The bone-related therapeutic agent is covalently bound to the spacer; or linked to the spacer via a physical interaction, such as a cyclodextrin-hydrophobic molecular enclosure complex, where the host molecules (cyclodextrin) are covalently bound to the spacer. The monomeric structure connected with the bone-related therapeutic agent via the spacer contributes from 0.1 to 20 mol % of the polymer backbone.

The spacer (S₂) between the bone-targeting moieties and the polymeric backbone may be biodegradable or non-degradable, which may or may not be similar to the structures of S₁ described above. The bone-targeting moieties (T) suitable for the present invention include but are not limited to: tetracycline, its derivatives and its analogs; bisphosphonates (such as alendronate), its derivatives and analogs; D-(glutamic acid)ₓ, L-(glutamic acid)ₓ, D-(aspartic acid)ₓ (such as D-Asp₈) and L-(aspartic acid)ₓ (x=2˜100); sialic acid; malonic acid; N,N-dicarboxymethylamine; 4-aminosalicylic acid, 5-aminosalicylic acid; antibodies, antibody fragments, peptides, etc. The bone-targeting moieties (T) are covalently bound to the spacer (S₂). The monomeric structure connected with the bone-targeting moieties via the spacer (S₂) will contribute from 0.1 to 95 mol % of the polymer backbone.

Optionally, the non-degradable spacer (S₃) between the bioassay label and the copolymer backbone is a covalent bond or any other chemical structure, which will not be cleaved under physiological environments or conditions. The bioassay labels (L) suitable for the present invention may include but are not limited to: tyrosine (¹²⁵I labeling), fluorescein isothiocyanate (microscopic visualization and histomorphometric analysis), etc. The bioassay labels (L) are covalently bound to the spacer (S₃). The monomeric structure connected with the bioassay label via the spacer contributes from 0 to 10 mol % of the copolymer backbone.

The optional biodegradable cross-linkage (C) suitable for the present invention can be a peptide structure represented by the formula:-Pep-Q-Pep-, wherein Pep is a peptide which may include but is not limited to the following sequences: Gly-Leu-Gly, Gly-Val-Gly, Gly-Phe-Ala, Gly-Leu-Phe, Gly-Leu-Ala, Ala-Val-Ala, Gly-Phe-Leu-Gly, Gly-Phe-Phe-Leu, Gly-Leu-Leu-Gly, Gly-Phe-Tyr-Ala, Gly-Phe-Gly-Phe, Ala-Gly-Val-Phe, Gly-Phe-Phe-Gly, Gly-Phe-Leu-Gly-Phe, and Gly-Gly-Phe-Leu-Gly-Phe; and Q is a linking group with a diamine structure. The monomeric structure connected to the biodegradable cross-linkage preferably contributes from 0 to 5 mol % of the copolymer backbone.

Usually, two types of strategies can be used for introducing functional moieties into the N-(2-hydroxypropyl)methacrylamide (HPMA) copolymers, (1) copolymerization of HPMA with polymerizable functional comonomers; and (2) direct conjugation of active ester containing HPMA copolymers with functional moieties, which preferably bear a primary amine group.

The following examples will enable those skilled in the art to more clearly understand how to practice the present invention. It is to be understood that, while the invention has been described in conjunction with the preferred specific embodiments thereof, that which follows is intended to illustrate and not limit the scope of the invention. Other aspects of the invention will be apparent to those skilled in the art to which the invention pertains. The following are the abbreviations used in the description:

(1) ACV, 4,4′-azobis(4-cyanopentanoic acid); (2) ATC, anhydrotetracycline hydrochloride; (3) DCC, dicyclohexylcarbodiimide; (4) DCM, dichloro methane; (5) DCU, dicyclohexyl urea; (6) DIPEA, diisopropylethyl amine; (7) DMF, N,N′-dimethyl formamide; (8) DMSO, dimethyl sulfoxide; (9) FPLC, fast protein liquid chromatography; (10) FITC, fluorescein isothiocyanate; (11) 9-Gly-ATC, N-(9-aminoanhydrotetracycline)glycyl amide; (12) HA, hydroxyapatite; (13) HOBt, 1-hydroxybenzotriazole; (14) HOSu, 1-hydroxy-succinimide; (15) HPMA, N-(2-hydroxypropyl)methacrylamide; (16) MA-FITC, N-methacryloylaminopropyl fluorescein thiourea; (17) MA-GG-ONp, N-methiacryloylglycylglycine p-nitrophenyl ester; (18) MA-GG-D-(Asp)₈, N-methacryloylglycylglycine D-(aspartYl)₈ amide; (19) MeOH, methanol; (20) Mₙ, number average molecular weight; (21) MPA, mercaptopropionic acid; (22) M_{w}, weight average molecular weight; (23) MWD, molecular weight distribution; (24) 9-NH₂-ATC, 9-aminoanhiydrotetracycline hydrochloride; (25) NHS, N-hydroxysuccinimide ester; (26) -ONp, p-nitrophenyl ester; (27) P-D-(Asp)₈-FITC, a copolymer of HPMA, MA-GG-D-(Asp)₈ and MA-FITC; (28) P-GG-ONp, a copolymer of HPMA, MA-GG-ONp; (29) P-GG-ONp-FITC, a copolymer of HPMA, MA-GG-ONp and MA-FITC; (30) P-Alendronate-FITC, a conjugate of P-GG-ONp-FITC with alendronate wherein alendronate is linked to the polymer via a Gly-Gly spacer; (31) P-ATC-Rhodamine, a conjugate of P-GG-ONp with 9-Gly-ATC and rhodamine cadaverine, wherein they are linked to the polymer backbone via a Gly-Gly spacer; (32) PHPMA, poly[N-(2-hydroxypropyl)methacrylamide]; (33) P-, HPMA copolymer backbone; (34) R. T., room temperature; (35) TC, tetracycline; and (36) TFA, trifluoroacetic acid.

### EXAMPLE 1

### Synthesis of 9-Gly-ATC

This example illustrates the synthetic process for the preparation of the novel N-(9-aminoanhydrotetracycline)glycyl amide (9-Gly-ATC) of the present invention.

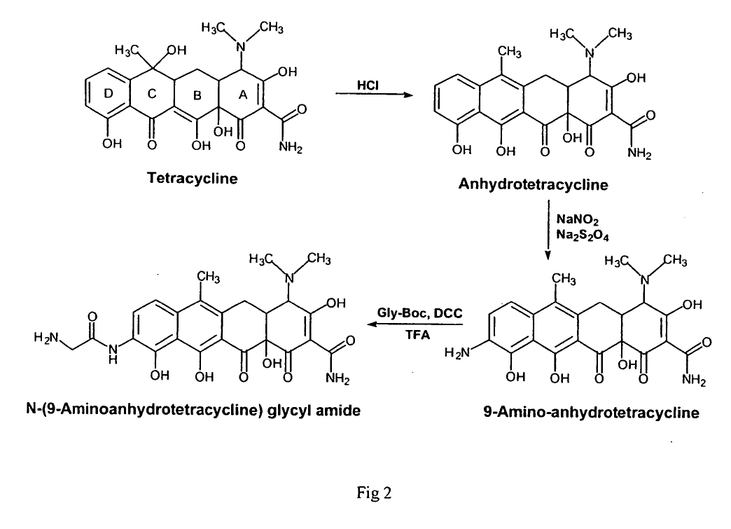

TC is an antibiotic which shows a strong binding to hard tissue. Perrin, D. D. (1965) Nature 208, 787-788. However, its native structure does not have the proper functional group, such as an amine, to allow its attachment to polymers. Therefore, certain chemical modifications must be made to the TC structure. As reported previously, the keto-enol ligand of rings B and C and the tricarbonylmethane grouping of ring A are essential for the binding of TC to hydroxyapatite. Myers, H. M. et al.(1983) Tissue Int. 35, 745-749. In order to retain the desired binding ability, modification of the TC molecule was carried out on the ring D (FIG. 2).

TC was first transformed into anhydrotetracycline; an amino group was then introduced into the D ring. Stoel, L. et al.(1976) J. Pharm. Sci. 65, 1794-1799; Menachery, M. D. & Cava, M. P. (1984) Can. J. Chem. 62, 2583-2584. Because of the low activity of the aromatic amine, a glycine was coupled to the D ring of the TC molecule in order to introduce a more reactive primary amine by the following procedure. Boc-glycine (192.7 mg, 1.1 mmol), 9-NH₂-ATC (514 mg, 1 mmol) and methyl morpholine (MM, 220 μL, 2 mmol) were dissolved in DMF (8 mL) and stirred at 0° C. for 1 h. DCC (227 mg, 1.1 mmol, in 2 mL DCM) was added to the solution and stirred for 1 h. The temperature of the solution was then raised to R.T. and stirred overnight. The resulting suspension was filtered. The solid (product and DCU) was washed three times with ethyl acetate and dried under vacuum. The Boc protection was removed with TFA. DCU was removed to yield 360 mg of 9-Gly-ATC (lyophilized), which is water-soluble.

### EXAMPLE 2

### Conjugation of 9-Gly-ATC to P-GG-ONp

9-Gly-ATC was conjugated to P-GG-ONp (a copolymer of MA-GG-ONp and HPMA) by the following procedure. Kope\check{c}ek, J., Ba ilová, H. (1973) Eur. Polym. J. 9, 7-14; Rejmanová, P., Labsk\dot{y}, J., Kope\check{c}ek, J. (1977) Makromol. Chem. 178, 2159-2168. P-GG-ONp (50 mg, [ONp]=2.9×10⁻⁵ mol) and 9-Gly-ATC (50 mg, 6.9×10⁻⁵ mol) were dissolved in DMF (1 mL). DIPEA (29 μL, 1.67×10⁻⁴ m) was added to the solution. The solution was stirred at R.T. overnight and then purified on an LH-20 column, a PD-10 column and a Superdex 75 column. The conjugate was dialyzed against water (MWCO 6˜8 kDa) and lyophilized to yield 40 mg of purified P-ATC.

### EXAMPLE 3

### Synthesis of P-ATC-Rhodamine

By a similar procedure as described in Example 2, P-ATC-Rhodamine was synthesized by conjugating 9-Gly-ATC and Rhodamine cadaverine together to P-GG-ONp.

### EXAMPLE 4

### Conjugation of Alendronate to P-GG-ONp-FITC

Alendronate bears a primary amine, which can be used for conjugation with active ester containing polymers. Its conjugation to copolymers was carried out in aqueous solutions. Alendronate (100 mg, 3.08×10⁻⁴ mol) was suspended in water (1 mL). While vigorously stirring, P-GG-ONp-FITC (50 mg, ONp=2.75×10⁻⁵ mol, in 200 μL of DMF) was added to the aqueous solution. Under constant monitoring of the pH of the solution, NaOH (0.2 M) was added to slowly raise the pH value to 7. After 1 hour, the pH was increased to 8. Afterwards, the pH was rapidly raised to 9, finishing the reaction. Free ONp was removed with PD-10 columns. The conjugate was then dialyzed against water (MWCO 6˜8 kDa). It was lyophilized to yield 36 mg of the titled product.

### EXAMPLE 5

### Synthesis of Polymerizable D-(Asp)₈ Derivative

Hexapeptides of aspartic acid have been reported as being used as bone-targeting moieties. Kasugai, S., et al. (2000) J. Bone Miner. Res. 15, 936-943. To ensure proper in vivo stability and stronger binding, an octapeptide of D-aspartic acid was used in the present study. Direct conjugation of D-(Asp)₈ to a HPMA copolymer could only be carried out in an aqueous solution. However, the conjugation ratio of the peptide was extremely low. To solve the problem, a polymerizable D-(Asp)₈ derivative was synthesized as follows.

Routine solid phase peptide synthesis of D-(Asp)₈ was initiated by loading D-(Asp-OtBu) (67 mg, 0.1 62mmol) onto a trityl chloride resin (300 mg, 0.324 mmol of -Cl, 50% loading). Chan, W. C. & White, P. D. (2000) In Fmoc Solid Phase Peptide Synthesis, A practical Approach. (Chan, W. C., White, P. D. Ed.) Oxford University Press Inc., New York, N.Y., pp 41-74. A stepwise procedure was followed until eight D-(Asp-OtBu) had been connected. After the NH₂ of the final D-Asp-OtBu was exposed with piperidine, MA-GG-ONp (260 mg. 0.810 mmol) and DIPEA (226 μL, 1.296 mmol) were added (in 1.5 mL of DMF). The resulting solution was transferred to a vial and rotated overnight. The resin was then washed and the product was cleaved with TFA. When the product was cleaved from the resin the carboxyls were also deprotected simultaneously. The product was fractioned with a Superdex 75 column on FPLC, dialyzed and lyophilized to yield about 70 mg of MA-GG-D-(Asp)₈.

### EXAMPLE 6

### Copolymerization of MA-GG-D-(Asp)₈ and HPMA

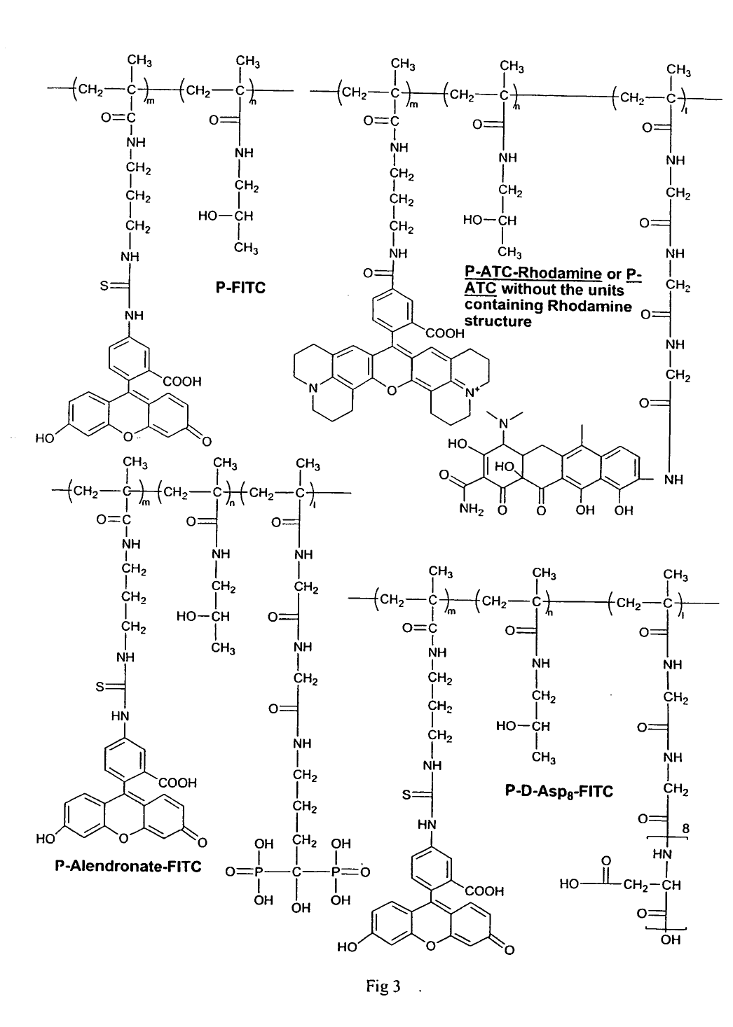

To synthesize a D-(Asp)₈ containing copolymer, HPMA (50 mg, 3.5×10⁻⁴ mol) and MA-FITC (2.5 mg. 4.6×10⁻⁶ mol) were dissolved in DMSO (0.5 mL) and mixed with the aqueous solution (1 mL) of MA-GG-D-(Asp)₈ (20 mg, 1.79×10⁻⁵ mol) and ACV (5.8 mg, 2.07×10⁻⁵ mol). The solution was then purged with N₂ and sealed in an ampoule to allow polymerization to occur. Polymerization was carried out at 50° C. for 18 h. The solution was then diluted and purified with PD-10 columns and dialyzed against water (MWCO 6˜8 kDa). The polymer was then further purified with FPLC (Superdex75). The polymer fraction was dialyzed, and lyophilized to obtain 44 mg of P-D-(Asp)₈-FITC. The characterization of all conjugates described above is summarized in Table 1. Their chemical structures are depicted in FIG. 3.

TABLE 1
Characterization of polymeric bone-targeting conjugates

| | | | Bone-targeting |
|---|---|---|---|
| | MW | Fluorochrome | Moiety Content |
| Conjugates | (kDa) | Content (mol/g) | (mol/g) |
| P-FITC | 25 | 3.80 × 10⁻⁵ | — |
| P-Alendronate-FITC | 26 | 3.86 × 10⁻⁵ | 4.94 × 10⁻⁴ |
| P-D-Asp₈-FITC | 43 | 3.82 × 10⁻⁵ | 7.62 × 10⁻⁵ |
| P-ATC | 26 | — | 2.00 × 10⁻⁴ |
| P-ATC-Rhodamine | 26 | 1.09 × 10⁻⁵ | 9.05 × 10⁻⁵ |

### EXAMPLE 7

### Assays for Bone-Targeting Capacities of the Conjugates of Table 1 in vitro

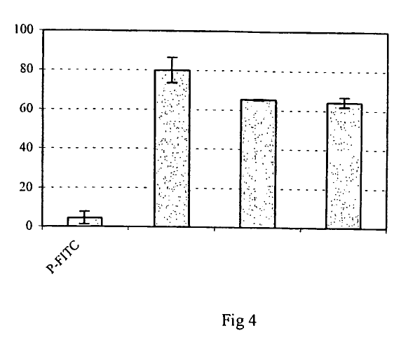

All conjugates in Table 1 were screened in vitro for their bone-targeting capacity by the following procedure. Conjugates were dissolved in phosphate buffered saline to give a concentration of 1 mg/mL. The conjugate solution (100 μL) and 100 μL of the same buffer were incubated with 5 mg of hydroxyapatite (HA, Bio-Gel HTP, DNA grade; BIO-RAD, Hercules, Calif.) for 1 h at R.T. The solution was then centrifuged. The UV absorbance at certain wavelengths (FITC, 490 nm; 9-Gly-ATC, 450 nm) of the supernatant was monitored with an ELISA plate reader. Background correction was applied. The data presented is the average of three samples. The binding efficiency is expressed as the percentage of conjugates bound to HA (FIG. 4).

As displayed in FIG. 4, all HPMA copolymer bone-targeting conjugates showed good binding to HA, while the HPMA copolymer itself (P-FITC, without a targeting moiety) showed only very low non-specific binding to HA. Among the three targeting moieties, D-Asp₈ demonstrated the highest HA binding potential, while the Alendronate and 9-Gly-ATC showed slightly lower values. Multivalent binding might contribute to the binding of the conjugates as well.

### EXAMPLE 8

### Assays for the Bone-Targeting Capacities of the Conjugates of Table 1 in vivo

The in vivo bone-targeting capacities of these conjugates were evaluated as follows. Balb/c mice (˜20 g, male, Charles River Laboratories, Inc., Wilmington, Mass.) were injected i.v. (in tail vain) with all FITC labeled conjugates at a FITC dose of 1.84×10⁻⁵ mol/kg. After 24 hours, all animals were sacrificed. The femur and tibia were isolated, fixed with formalin, dehydrated with acetone, embedded in poly(methyl methacrylate) and sliced (100 μm) for fluorescence microscopic analysis.

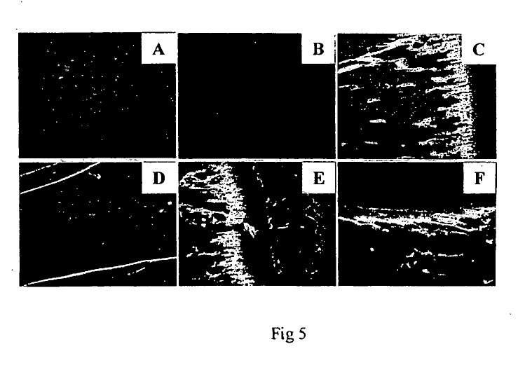

As shown in FIG. 5, no autofluorescence was observed in the animals injected with saline. For those injected with P-FITC, no fluorescence was observed either. Interestingly, all FITC labeled bone-targeting conjugates showed a very bright FITC label throughout the bone. The epiphysis, metaphysis and diaphysis were marked with fluorescence. A detailed examination indicated the strongest labeling in the metaphyseal region next to the epiphyseal plate and the metaphyseal funnel. Plus, both the endosteum and the periosteum of the diaphyseal shaft were marked with clear lines of the FITC label. In addition, a high intensity of FITC label was observed in the bones of the axial skeleton including the vertebra and mandibles. Presumably, these bone-targeting delivery systems prefer to accumulate in the growth sites of bone, where sufficient blood supply is available.

To further understand the biodistribuition of the bone-targeted conjugate, vital organs (liver, heart, lung, intestine, kidney, spleen) from the animals injected with P-(D-Asp₈)-FITC were isolated and processed for histological analysis. All histological samples were analyzed with a semi-quantitative fluorescence image analysis system (BioQuant, NovaPrime-XP). There was no detectable fluorescence in spleen, heart, lung, intestine and bone marrow, while the bone surfaces were saturated with fluorescence signal of the injected P-(D-Asp₈)-FITC. Minor fluorescence (˜5% compared to that found in bone) was in the kidney and liver. In the control group, P-FITC (no targeting) injected animals were also evaluated with the same method. We did not detect significant fluorescence in any bones and there was no fluorescence in other organs except for a minor signal in liver and kidney.

Another quantitative biodistribution study was performed to estimate the amount of P-(D-Asp₈)-FITC in the long bones (four limbs) when compared with P-FITC. Twenty-four hours after injection, the bones were isolated and decalcified with EDTA for 72 hrs. The tissue was then homogenized and filtered. The filtrate was further diluted with buffer (pH=10, 0. 1% surfactant) and the fluorescence of the FITC was measured. The preliminary results showed that 12.7% of the original dose of the P-(D-Asp₈)-FITC injected was recovered from the long bones of the extremities, while only 3.2% of the original dose of the P-FITC injected could be found in the limbs of the animal. Similar results were also observed with conjugates using alendronate as the targeting moiety. Apparently, both D-Asp₈ and alendronate are potent bone-targeting moieties, and can effectively direct the polymeric carrier to the skeleton.

With the dose administrated, all animals injected with the FITC labeled conjugates remained normal and active until they were sacrificed.

Therefore, the present invention provides bone-targeting polymeric drug delivery systems based on HPMA copolymers wherein tetracycline derivatives, alendronate and an octapeptide of D-aspartic acid were used as bone-targeting moieties by either direct conjugation or copolymerization. In vitro and in vivo studies indicate that Alendronate and D-Asp₈ based conjugates are very good candidates for the bone-targeted drug delivery.

It is to be understood that the above-referenced embodiments are only illustrative of application of the principles of the present invention. Numerous modifications and alternative arrangements can be devised without departing from the spirit and scope of the present invention. While the present invention has been shown in the examples and is fully described above with particularity and detail in connection with what is presently deemed to be the most practical and preferred embodiment(s) of the invention, it will be apparent to those of ordinary skill in the art that numerous modifications can be made without departing from the principles and concepts of the invention as set forth in the claims.

1. A water-soluble bone-targeting drug delivery system comprising a water-soluble copolymer backbone (P) which is linked, via a first spacer (S₁), with a bone-related therapeutic agent or a drug (D), via a second spacer (S₂), with a bone-targeting moiety (T) and, via a third spacer (S₃), with a bioassay-label (L), and wherein said copolymer comprises 5.0 to 99.0 mol% of monomeric units selected from the group consisting of N-(2-hydroxypropyl)methacrylamide, N-(2-hydroxyethyl)methacrylamide, N-isopropylacrylamide, acrylamide, N,N-dimethylacrylamide, N-vinylpyrrolidone, vinyl acetate, 2-methacryloxyethyl glucoside, acrylic acid, methacrylic acid, vinylphosphonic acid, styrenesulfonic acid, maleic acid, 2-methacryloxyethyltrimethylammonium chloride, methacrylamidopropyltrimethylammonium chloride, methacryloylcholine methyl sulfate, N-methylolacrylamide, 2-hydroxy-3-methacryloxypropyltrimethylammonium chloride, 2-methacryloxyethyltrimethylammonium bromide, 2-vinyl-1-methylpyridinium bromide, 4-vinyl-1-methylpyridinium bromide, ethyleneimine, (N-acetyl)ethyleneimine, (N-hydroxyethyl)ethyleneimine and allylamine.

2. The delivery system according to claim 1 wherein the molecular weight of the water-soluble copolymer backbone (P) is within the range of 1 to 500 kDa.

3. The delivery system according to claim 1, further comprising a bioassay label (L) which is attached to the copolymer backbone via a third spacer (S₃).

4. The delivery system according to claim 1, wherein the bone targeting moiety and bone-related therapeutic agent containing copolymer is cross-linked via a biodegradable cross-linkage (C).

5. The delivery system according to claim 1 wherein the bone-related therapeutic agent is a member selected from the group consisting of cathepsin K inhibitors, metalloproteinase inhibitors, prostaglandin E receptor agonists, αvβ3 antagonists, anabolic agents, parathyroid hormone, statins, therapeutic peptides and therapeutic metal ions.

6. The delivery system according to claim 1, wherein the bone-targeting moiety is a member selected from the group consisting of tetracycline, its derivatives and analogs; alendronate, its derivatives and analogs; D-(glutamic acid)ₓ, L-(glutamic acid)ₓ, D-(aspartic acid)ₓ (such as D-Asp₈)ₓ and L-(aspartic acid), wherein x is an integer of 2˜100; sialic acid; malonic acid; N,N-dicarboxymethylamine; 4-aminosalicylic acid, 5-aminosalicylic acid; antibodies and peptides.

7. The delivery system according to claim 1, wherein the spacers S₁ and S₂ are biodegradable structures represented by one of the following: 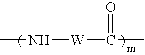
wherein W is the portion of an amino acid other than an NH₂ or COOH group, said amino acid having an L-configuration and being selected from among all the essential amino acids, and m is an integer from 1 to 10; 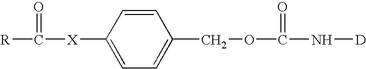
wherein R may be a peptide structure described above, which is directly connected to the polymer backbone and D represents the bone-related therapeutic agent of which the amine group(-NH-) is a part; and X can be O or NH; and 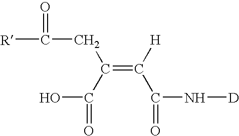
wherein R′ may be a C₀ to C₁₀ alkyl amino, aryl amino, a C₀ to C₁₀ alkyl amino or aryl oxy, which is directly connected to the polymer backbone and D represents the bone-related therapeutic agent of which the amine group(-NH-) is a part.

8. The delivery system according to claim 3, wherein the spacers S₁, S₂ and S₃ are non-degradable and can be a covalent bond or a chemical structure which cannot be cleaved under physiological environments or conditions.

9. The delivery system according to claim 1, wherein the water-soluble copolymer backbone is cross-linked by peptide structure -Pep-Q-Pep- wherein Pep is a member selected from the group consisting of Gly-Leu-Gly, Gly-Val-Gly, Gly-Phe-Ala, Gly-Leu-Phe, Gly-Leu-Ala, Ala-Val-Ala, Gly-Phe-Leu-Gly, Gly-Phe-Phe-Leu, Gly-Leu-Leu-Gly, Gly-Phe-Tyr-Ala, Gly-Phe-Gly-Phe, Ala-Gly-Val-Phe, Gly-Phe-Phe-Gly, Gly-Phe-Leu-Gly-Phe, and Gly-Gly-Phe-Leu-Gly-Phe, and Q is a linkage group of diamine structure.

10. A tetracycline derivative, 9-Gly-ATC, having the structure as the following: 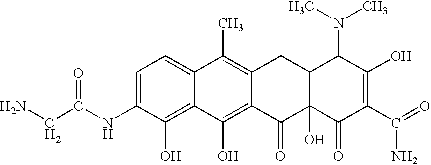
wherein said tetracycline derivative can be used as a bone-targeting agent or a novel antibiotic agent.

11. A water-soluble bone-targeting drug delivery system represented by the following formula: 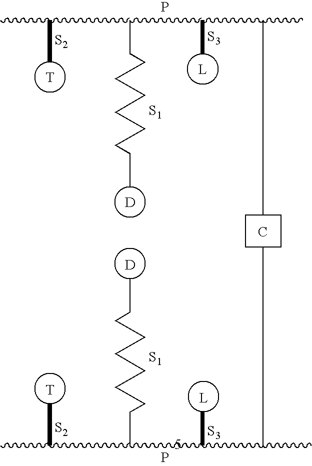
wherein D is a bone-related therapeutic agent bonded to a water soluble inert polymer backbone (P) via a first spacer (S₁) which may be biodegradable or non-biodegradable; T is a bone-targeting molecule covalently bound to the polymer backbone (P) via biodegradable or non-degradable spacer (S₂); L is an optional bio-assay label covalently bonded to the polymer backbone (P) via a non-degradable third spacer (S₃) which can be the same or different than S₁ or S₂ when they are non-degradable; and C is an optional biodegradable cross-linkage between two polymer chains (P).

12. A pharmaceutical formulation comprising the water-soluble bone-targeting drug delivery system according to claim 1 a biocompatible excipient selected from the group consisting of water, saline, dextrose, glycerol, ethanol; and an auxiliary substances selected from the group consisting of wetting or emulsifying agents and buffers.

13. The pharmaceutical formulation of 12 is formulated as a solution, a suspension, an emulsion or other liquid forms, tablets, capsules or other solid forms.

14. The pharmaceutical formulation of 13 is suitable for injection or oral administration, transdermal drug delivery, transmucosal drug delivery, inhalation, or controlled release implantation.
